(19) United States
(12) Patent Application Publication
(10) Pub. No.: US 2005/0060039 A1
(43) Pub. Date: Mar. 17, 2005

(54) Shoulder glenoid prosthesis with method and tools for implanting it

(76) Inventor: Jean-Maxwell Cyprien, Geneva (CH)

Correspondence Address: Jean-Maxwell Cyprien, 11, rue du Conseil-General, GENEVA, 1205, CH

(21) Appl. No.: 10/659,515

(22) Filed: Sep. 11, 2003

(51) Int. Cl. A61F 2/40

(52) U.S. Cl. 623/19.13

(57) ABSTRACT

A glenoid plastic prosthesis 100 for cementation is described, comprising a pear-shaped body having a concave articular face 26 and a convex face with a horizontal keel 28 and threaded pegs 34. Indentations 30 on the keel and the convex surface of the implant follow a generally fractal pattern in order to increase the implant cement interface. Tools are provided such as
a) A glenoid-marking tool 58 used to stamp into the glenoid subchondral bone patterns of indentations of a generally fractal nature and b) A glenoid indentation tool used to make indents in walls of the glenoid cavity for the keel in order to increase the bone-cement interface.

### CROSS-REFERENCES TO RELATED APPLICATIONS

U.S. PATENT DOCUMENTS

| | | |
|---|---|---|
| 5,080,673 | Burkhead et al | Jan. 14, 1992 |
| 5,489,310 | Mikhail | Feb. 16, 1996 |
| 5,593,448 | Dong | Jan. 14, 1997 |
| 5,723,285 | Cyprien, et al | Mar. 3, 1998 |
| 5,928,285 | Bigliani, et al | Jul. 27, 1999 |
| 6,379,386 B1 | Resh et al | Apr. 30, 2002 |
| 20030065405 A1 | Amrich, et al | Apr. 03, 2003 |
| 6,565,602 B2 | Rolando et al. | May 20, 2003 |

Foreign Patent Document:
Non Patent References:
1. Anglin C. Wyss U P, Pichora D R Mechanical testing of shoulder prosthesis and recommendations for glenoid design J. Shoulder and Elbow Surg. 2000, 9: 323-31
2. Checroun A J, MD, Hawkins C, MD, Kummer F J, PhD, Zuckerman J D, MD Fit of current glenoid component designs: An anatomic cadaver study J J. Shoulder and Elbow Surg. 2002•Volume 11•Number 6
3. Nyffeler R. W., Anglin C., Sheik R., Gerber C. Influence of peg design and cement mantle thickness on pull-out strength of glenoid component pegs J. Bone and Joint Surg. 2003, 85-B: 748-752

### FEDERALLY SPONSORED RESEARCH

Not applicable

### SEQUENCE LISTING OR PROGRAM

Not applicable

### BACKGROUND OF THE INVENTION

Replacement of diseased or injured joints is common practice in orthopedic surgery. The replacement of a shoulder joint either for arthritic disease or for fracture by an artificial device can restore function in many cases.

However, total shoulder prosthetic replacement is a procedure fraught with difficulties. Among those, loosening of the glenoid implant is a most common complication. Radiolucent lines around the prosthesis are even more frequent. They can be present without pain or clinical signs of shoulder dysfunction but when progressive, they are indicative of loosening.

The causes of loosening are multifactorial. They include debris-induced osteolysis in which the role of the osteoclast lineage has been evoked, deficiency of rotator cuff function, improper design resulting in eccentric loading of the implant.

In designing glenoid prosthesis, a number of factors must be taken into account. Among them are the forces exerted over the inferior-superior axis, and the anterior-posterior axis. Those forces are responsible for eccentric loading of the implant. The inferior-superior forces, for instance, can induce the so-called the rocking-horse phenomenon in which of the implant is progressively pulled out of the bone. The other forces are either beneficial like the compressive component on the glenoid or inconsequential.

The design of the glenoid prosthesis must follow as much as possible the anatomy of the bone. Human glenoids have in general a pear-shaped form that the design should endeavor to reproduce. A rectangular prosthesis cemented on a pear-shaped glenoid will sustain stresses leading to an unseating of the implant. Flat-backed glenoid implants have been shown experimentally to resist less easily shearing forces and eccentric loading than a curved-backed glenoid.

Available glenoid implants are either all-polyethylene or metal-backed. They can be provided for cementation or with bone ingrowth structure. Cemented glenoid prostheses have either a keel or a number of pegs or both. Glenoid prostheses with keel seem to resist slightly less to experimental loosening forces than prostheses with pegs. Currently available glenoid prostheses with keel, like U.S. Pat. No. 5,928,285, have that keel in line with the greater axis of the implant or, like U.S. Pat. No. 6,379,386 B1, parallel to that axis. This is not the optimal position to resist to the stresses being inflicted on the glenoid implant by the inferior-superior forces. Stress=σ=F/A, F=applied forces, A=cross-sectional area. In this situation, the ratio of force acting tangentially on a limited area results in increased strain at the interfaces.

The problem is to reduce the stresses on the fixation of the glenoid whether it is by peg or by keel. Ways to reduce those stresses on the fixation of a glenoid implant is either to diminish the forces being applied, which are hardly compatible with a normal shoulder function, or to augment the area on which they are applied. One way to augment the area on which they are applied is to make the keel perpendicular to the inferior-superior forces.

Loosening of implants intervenes sometimes at the implant-cement interface but most often it occurs at the bone-cement.

Existing cemented glenoid implants have a high rate of radiolucent lines at the bone-cement interface that can be a foreboding to loosening. To diminish or altogether eliminate those lines, implant-cement and bone-cement interfaces must be rendered capable of withstanding stresses occasioned by activities of daily living. Other avenues like the use of certain molecules regulating negatively the activity of the osteoclast lineage might also in this regard help in the future.

Improving the implant-cement interface and the bone-cement interface capability to weather loosening stresses can be done by increasing their overall contact surface. In this regard, fractal geometry is of a tremendous help.

Fractal geometry comprises an alternative set of geometric principles conceived and developed by French mathematician Benoit B. Mandelbrot. Those geometric principles have been used in various fields including the medical field; see for instance U.S. Pat. No. 6,565,602 by Rolando et al. or the U.S. Pat. No. 6,287,296 by Seiler, et al among others.

Fractals are geometrical figures that possess self-similarities also called invariance of scale. This means they have the same structure either on a large or on a small scale. Such figures are produced as a limit configuration of a sequence of fragments of curves. From each of these curves, the next is obtained following a defined rule. One example is the replacement of each side by a predetermined fragmentary called generative line or generator. The Koch's curve is generated by taking out one segment of an object and by replacing it by a number of segments whose length is equal to that of the removed segment.

Fractals can be generated based on their property of self-similarity by means of a recursive algorithm or by various initiators and generators.

One important aspect of fractal figures is their overall increase of length with the number of iterations. This is what Benoit Mandelbrot was expressing by saying that the length of a coastal line could be infinite depending on the scale on which it is measured.

This remarkable attribute is of interest to the present invention. By etching at a predetermined depth a fractal figure on the medial aspect of a glenoid implant destined for cementation, the implant-cement interface is greatly increased. By the same token, engraving into the glenoid bone at a predetermined depth a fractal figure by means of a specific tool significantly increases the bone-cement interface.

To illustrate this point, let us consider a curve with 5 segments of 10 mm of length (FIG. 1). Now each segment is replaced with the entire object scaled down to the length of a segment (FIG. 2). Next, each segment of the new object is replaced by the new object again reduced to the appropriate scale (FIG. 3).

The first curve was 50 mm of length (5×10 mm). The second was 82.25 mm of length (25×3.33 mm). The last curve was 231.25 mm (25×25×0.37 mm). The overall length increases as the length of the individual segment diminishes. This simple fractal structure is called is a Koch curve starting with five segments.

Now let us consider a given surface in which the above curves dig a trench, 3 mm deep and 1 mm wide. The total surface of the walls of the trough will be 300 mm (50 mm×3 mm×1 mm×2) for the first object. When we do the same iterations as described above, the surface is 493.50 mm² after the first iteration (82.25 mm×3 mm×1 mm×2), which is a 64.5% increase. After the second iteration the total surface will be 1387.50 mm² (231.25 mm×3mm1 mm×2). This means that after only 2 iterations and for a limited area, the surface increase is of 362.5%.

The present invention eliminates the disadvantages of ordinary vertical keel glenoids, flat back glenoids and smooth back glenoids. The keel is horizontal instead of being vertical; the convex surface of the implant takes advantage of fractal geometry and the holding power of threaded pegs is used. Like in U.S. Pat. No. 5,723,285 by Cyprien, et al, this invention has a non-conforming articular surface with 2 radiuses, the lower curvature being the largest.

Method to implant the prosthesis includes use of specific tool like a pear-shaped sizing drill guide, a glenoid-marking tool and a glenoid-indentation tool.

The holes drilled for the threaded pegs are made larger than the actual diameter of the pegs in order to accommodate the cement and a tap is used to cut threads into the walls of the holes. The cutting of those threads is of importance. It has been shown that smooth pegs pull out easily from the cement, while threaded pegs pull out with more difficulties and applied forces. The same thinking is valid for also creating threads in the walls of the peg's holes in which the cement will have a better hold.

The bony cavity for the keels is also larger than the actual diameter of the keel in order to accommodate cement. This cavity is created by three drill holes and by progressive compaction of the bone of the central glenoid. A glenoid indentation tool is used to make a number of cuts into the walls of the cavity in order to enhance, also at this level, the bone-cement interface.

### SUMMARY OF THE INVENTION

The invention relates to prostheses to be fixed in the bone of the glenoid part of a human scapula in total shoulder replacement. The objective of this invention is an implant capable of withstanding the loosening forces on glenoid implants after the surgery.

A pear-shaped curved-back glenoid prosthesis for cementation is described. The medial aspect of the implant has a horizontal keel, which is a keel perpendicular to the long axis of the body of the prosthesis. A threaded peg is placed above, and another threaded peg below the keel at equal distance from the center point on the inferior-superior axis. The medial aspect, as well as the horizontal keel has indented reticular structure defined following fractal geometry.

A sizing drill guide with head of different sizes is used to determine the dimensions of the glenoid and subsequently to drill the appropriate holes for the keel and for the pegs. The angle between the head of the sizing drill guide can be modified to accommodate the surgeon. After an adequate reaming to expose the subchondral bone, fractal figures of predetermined depth are created with a glenoid-marking tool. The cavity for the horizontal keel is prepared by bone compaction. Next, a number of indentations are done within the cavity walls with a glenoid indentation tool. Threads are cut in the holes for the pegs with a tap.

An improved bonding between the medial aspect of the implant and the cement on one hand and the between the cement and the bone on the other is achieved. Both interfaces are maximized, thereby optimizing the hold of the implant in the bone.

### DESCRIPTION OF THE SEVERAL VIEWS OF THE DRAWING

Note

Details of design, manufacture and procedure may be modified without departing from the spirit, intent and scope of the invention described herein.

Embodiments of the invention are described in more detail in the next section using the drawings. There are shown in:

FIG. 1: the initiator

FIG. 2: the generator

FIG. 3: a partial fractal curve after the second iteration.

FIG. 4-A: a front elevational view of the lateral aspect of the glenoid implant

FIG. 4-B: a front perspective view of the glenoid prosthesis

FIG. 5: a side elevational view of the glenoid implant

FIG. 6: a rear perspective view of the medial aspect of the implant.

FIG. 7: a top elevational view of the implant

FIG. 8: a side elevational view of the size-drill guide.

FIG. 9: a front elevational view of the size-drill guide in position.

FIG. 10-A: a side elevational view of the glenoid marking-tool.

FIG. 10-B: a front elevational view of the glenoid marking-tool.

FIG. 11: a sectional view of the glenoid bone showing of the cavity indentation tool in the cavity of glenoid for the keel.

FIG. 12: a sectional view of the glenoid bone showing of the cavity indentation tool open in the cavity of glenoid for the keel.

FIG. 13: a front elevational view of the articular aspect of the glenoid bone together with openings for the keel and the pegs together with a front perspective view of the prosthesis engaging in those apertures.

### DETAILED DESCRIPTION OF THE DRAWINGS

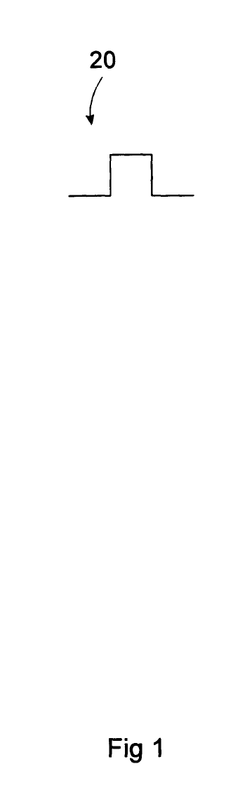

FIG. 1: This figure is the initiator 20. It consists of a segmented line having two ends, each segment being of equal length and connected to at least one adjacent segment.

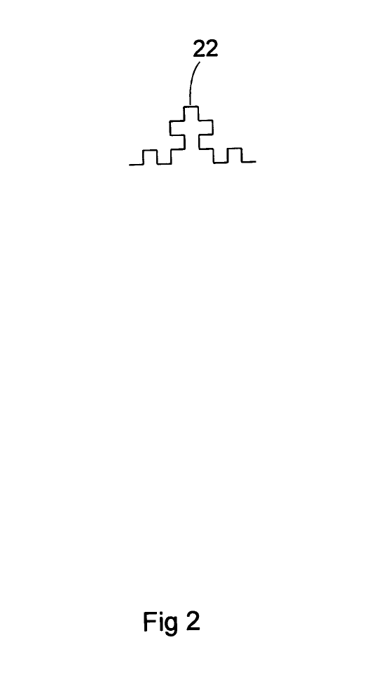

FIG. 2: This figure is the generator 22. It is obtained by replacing each segment of the initiator 20 by the initiator 20 reduced to the appropriate scale.

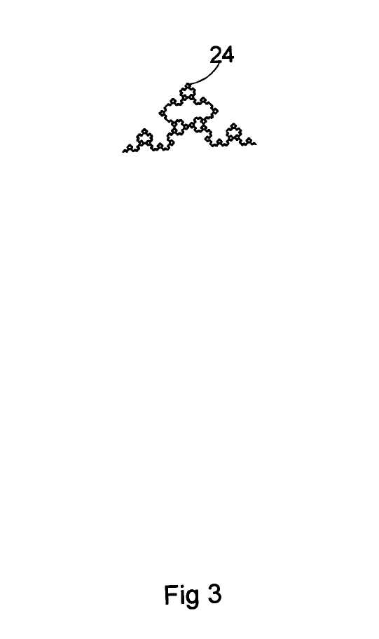

FIG. 3: a partial fractal FIG. 24 obtained by replacing each segment of the generator 20 by a scaled down version of the generator 20

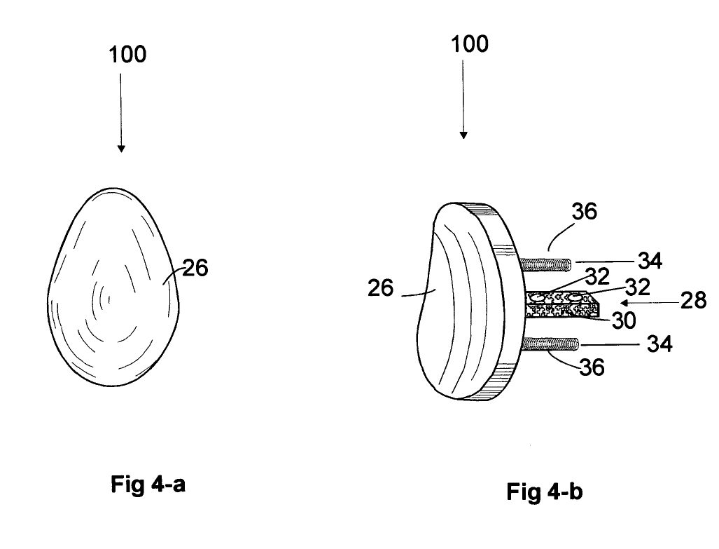

FIG. 4-A: a front elevational view of the prosthesis 100 showing its concave lateral articulating aspect 26.

FIG. 4-B: a perspective view of the implant 100 showing its articular surface 26 and the horizontal keel 28 with fractal indentations 30 and holes 32 and the pegs 34 with their threads 36.

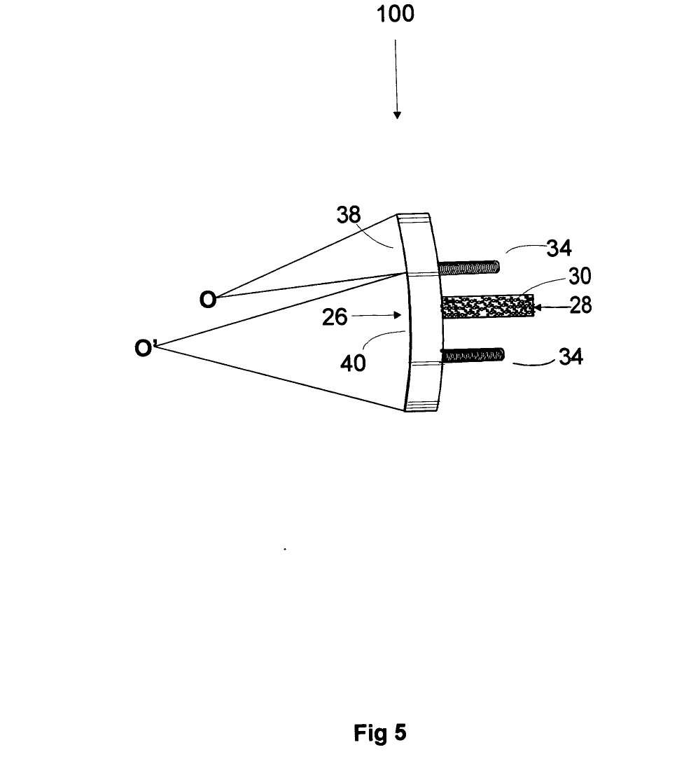

FIG. 5: a side elevational view of the implant 100 with an upper curvature 38 materialized by an arc of a circle with a center O and a lower curvature 40 materialized by an arc of a circle with a center O′. A side view of the horizontal keel 28 with one peg 34 above and one peg 34 below the keel 28.

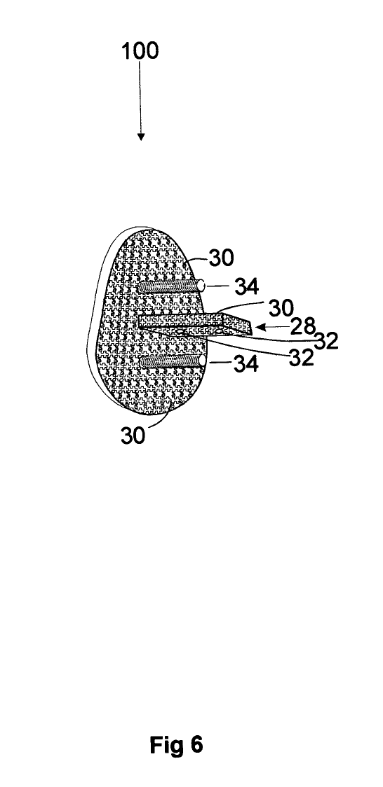

FIG. 6: a rear perspective view of the convex aspect of the implant 100 showing the horizontal keel 28 with transfixing holes 32. One peg 34 is placed above and one below the keel 28. The fractal indentations 30 are visible on the implant 100 including the keel 28.

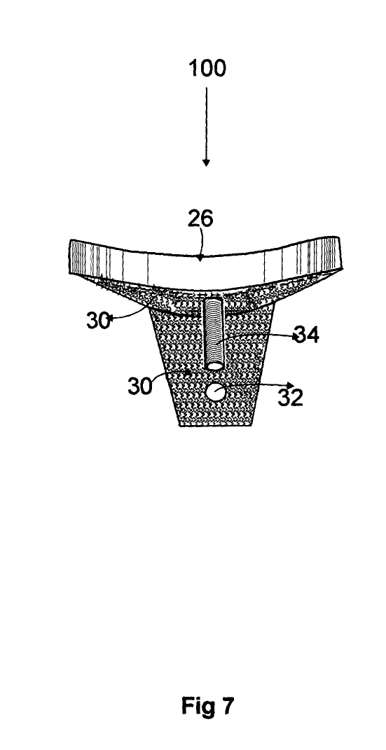

FIG. 7: a top elevational view of the implant 100 with the horizontal keel 28, a peg 34, a hole 32 and the fractal indents 30 on the implant 100

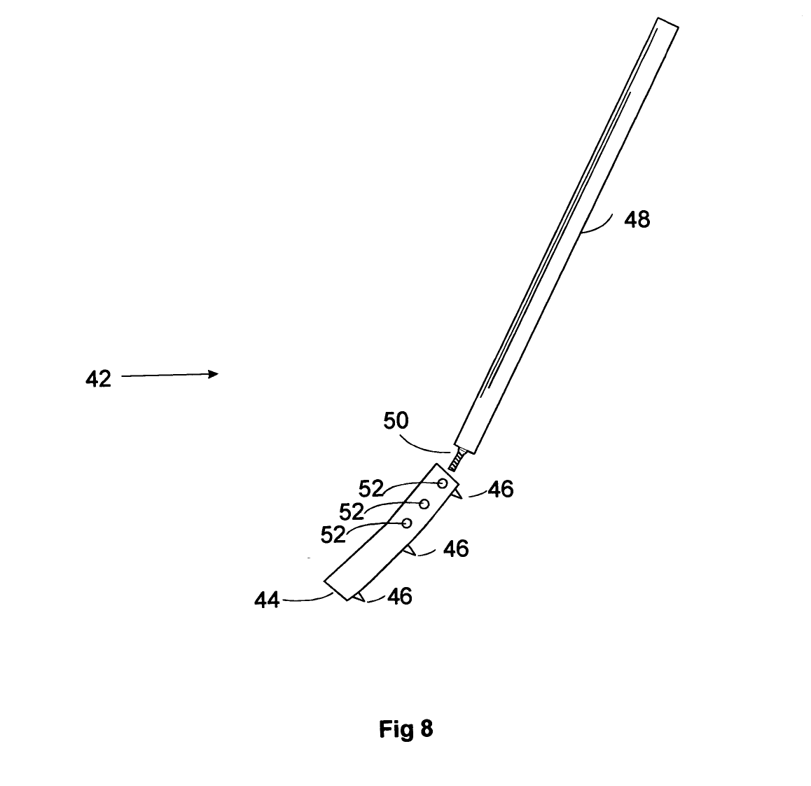

FIG. 8: a side elevational view of the sizing drill guide 42 with the head 44, the handle 48 with a threaded end 50 for fixation on the head 44. On each side of the head 44, there are three holes 52, in which the threaded end 50 of the handle can be fixed, depending of the surgeon preference, and a plurality of spikes 46 to stabilize the sizing drill guide in the glenoid before drilling.

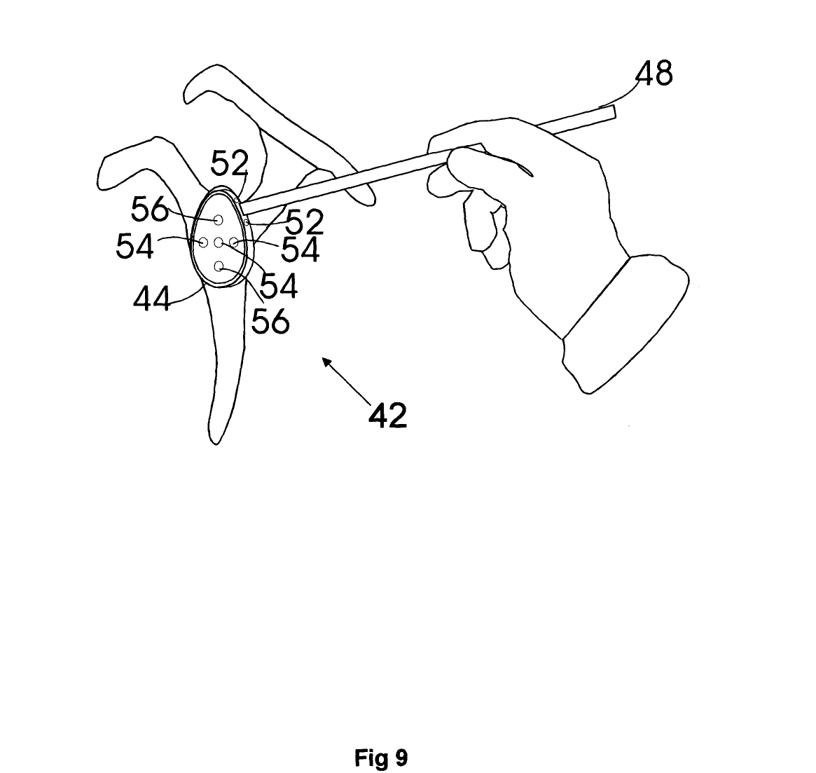

FIG. 9: a front elevational view of the sizing drill guide 42 in position with the head 44 of the guide, a handle 48. The head 44 has three holes 54 on a horizontal line, destined for the cavity 72 of the keel 28, and two drill holes 56 for the peg 34 of implant 100.

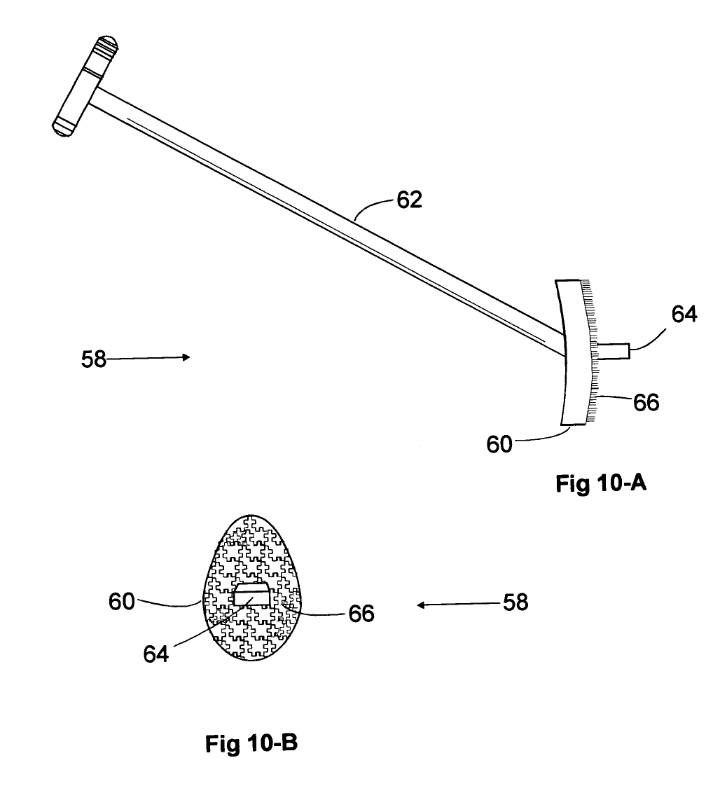

FIG. 10-A: a side elevational view of the glenoid-marking tool 58 showing a handle 62, a head 60 with a cutting blades 66 and a centering tip 64 for introduction in the cavity 72 for the keel 28.

FIG. 10-B: a front elevational view of the glenoid-marking tool 58 showing a head 60 with a centering tip 64 and cutting blades 66 on a substantially reticular pattern.

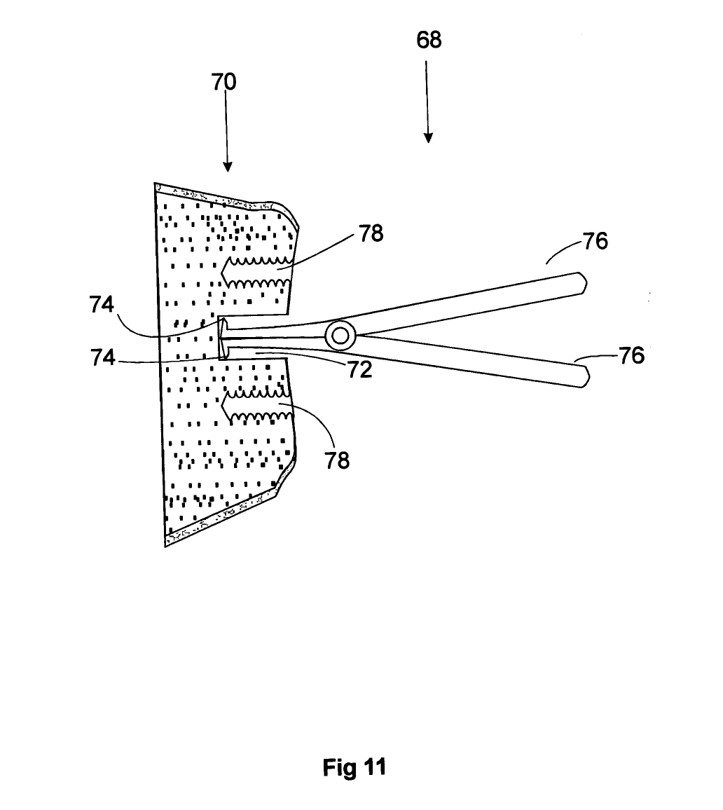

FIG. 11: a sectional view of the glenoid bone 70 along its major axis showing taped holes 78 destined for the pegs 34, a cavity 72 destined for the horizontal keel 28 and the cavity indentation tool 68 with blades 74 and handles 76.

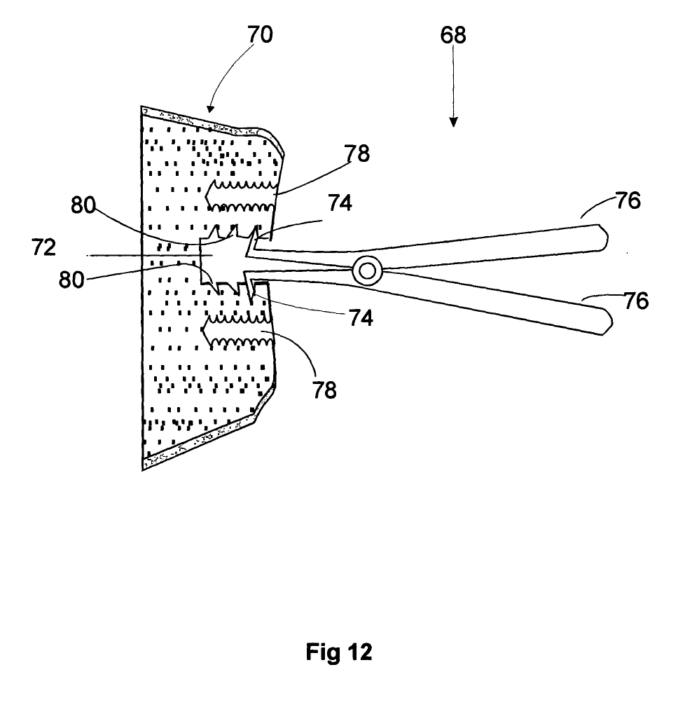

FIG. 12: a sectional view of the glenoid bone 70 along its major axis showing taped holes 78 destined for the pegs 34, a cavity 72 destined for the horizontal keel 28 and the cavity indentation tool 68 open with the blades 74 and the indentations 80 made by the tool 68.

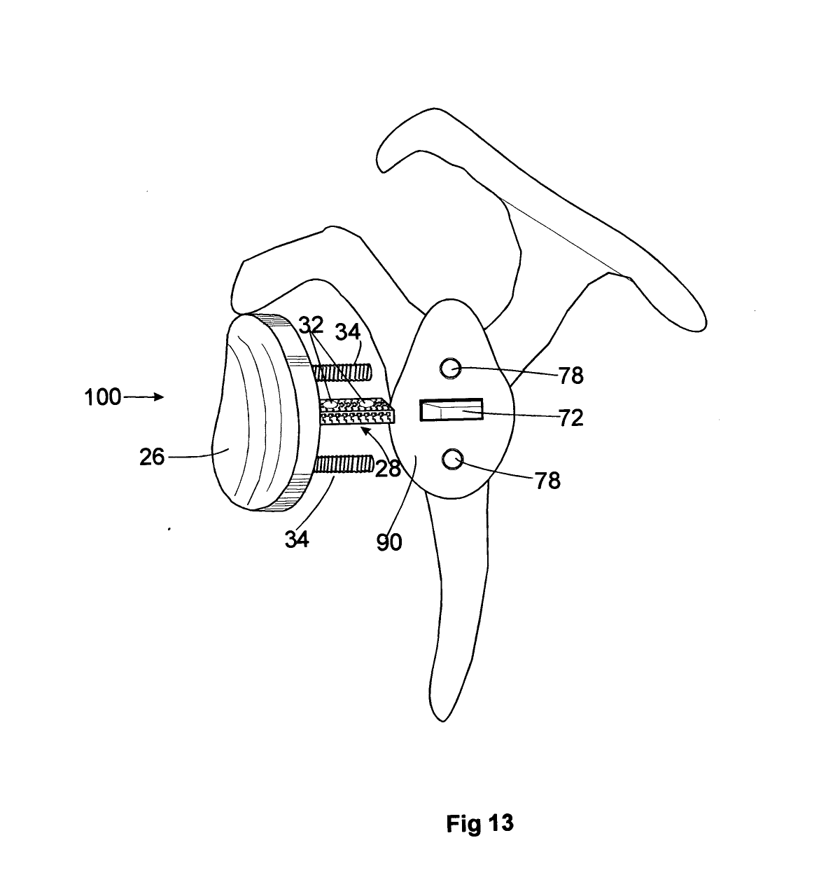

FIG. 13: an elevational view of the lateral aspect of the glenoid bone 90 with the cavity 72 for the horizontal keel 28 and the holes 78 for the pegs 34 and an oblique view of the implant 100 showing its articular surface 26 and the horizontal keel 28, the pegs 34, the prosthesis being positioned for implantation.

1. A glenoid plastic prosthesis for cementation comprising a pear-shaped body having a major axis extending along the largest diameter of said pear-shaped body, said body having a concave articular surface facing laterally and an opposing convex medial surface with a keel extending from said convex medial surface, said keel having a plane of symmetry perpendicular to the major axis of the body, having above and below it one or more peg

2. Said concave articular surface of the prosthesis of claim 1 having one or more radiuses on the sagittal plane and one or more radiuses in the transverse plane defining a non constraining articular surface for a humeral head.

3. Said convex medial surface and said keel having a substantially reticular indentation on its surface, means to enhance the implant cement interface

4. Said reticular indentation being alternatively defined by fractal geometry.

5. Said keel being substantially rectangular in shape and having a plurality of holes allowing cement penetration.

6. Said keel being substantially trapezoidal in shape and having a plurality of holes allowing cement penetration.

7. The glenoid plastic implant of claim 1 wherein one or more threaded pegs are above the keel and one or more threaded pegs are below the keel.

8. A method and tools for implanting the prosthesis of claim 1 comprising
a. a precise exposure of the bony margins of the glenoid and of its subchondral bone by predetermined means;
b. a pear-shaped size drill guide with changeable head sizes, means to evaluate the dimensions of said glenoid and to drill holes in said glenoid;
c. a glenoid-marking tool;
d. and a glenoid indentation tool.

9. Said drill holes being horizontally disposed, preparation for a cavity for said keel.

10. Said cavity for the keel being created by connecting the horizontal drill holes and by gradually compacting the bone of the central glenoid with predetermined means thereby increasing the density and resistance of its walls.

11. In another embodiment, drill holes being above and below said cavity for the keel.

12. Said drill holes being destined for said threaded pegs

13. The drill holes for the threaded pegs being of a larger diameter than that of the pegs

14. The drill holes for the threaded pegs having internal threads cut with a tap.

15. A pear-shaped convex marking tool with a handle and a plurality of blades being disposed on a substantially fractal pattern, means to make indents of predetermined depth in the glenoid bone after exposition of its subchondral bone by predetermined means.

16. In yet another embodiment, indentations of predetermined depth being made in the walls of the cavity in the glenoid for the horizontal keel with a glenoid indentation tool.

17. Whereby enhancing the hold of the implant in the bone after cementation.
